(19) United States
(12) Patent Application Publication
(10) Pub. No.: US 2022/0340312 A1
(43) Pub. Date: Oct. 27, 2022

(54) METHODS, SYSTEMS, AND COMPUTER PROGRAM PRODUCT FOR DISPENSING DRUG PRODUCT IN A DRUG PRODUCT PACKAGING SYSTEM USING ROUND-ROBIN DRAW DOWN FROM HOLDING CANISTERS

(71) Applicant: PARATA SYSTEMS, LLC, Durham, NC (US)

(72) Inventor: Brian Bishop, Wonder Lake, IL (US)

(21) Appl. No.: 17/660,045

(22) Filed: Apr. 21, 2022

Related U.S. Application Data
(60) Provisional application No. 63/179,738, filed on Apr. 26, 2021.

(51) Int. Cl. B65B 5/10; B65B 9/04; G06Q 10/08

(52) U.S. Cl. CPC B65B 5/103; B65B 9/045; G06Q 10/087

(57) ABSTRACT

A method includes generating an order for a drug product packaging system to dispense a quantity of drug product into respective ones of a plurality of pouches; associating a plurality of canisters in the drug product packaging system with each other as a group, each of the plurality of canisters containing the drug product; and operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

### RELATED APPLICATION

The present application claims priority from and the benefit of U.S. Provisional Application No. 63/179,738, filed Apr. 26, 2021, the disclosure of which is hereby incorporated herein by reference in its entirety.

### BACKGROUND

The present disclosure relates generally to the packaging of drug products, and, in particular, to methods, systems, and computer program products for packaging drug products using a drug product packaging system.

Drug product packaging systems may be used in facilities, such as pharmacies, hospitals, long term care facilities, and the like to dispense medications to fill prescriptions. These drug product packaging systems may include systems designed to package medications in various container types including, but not limited to, pouches, vials, bottles, blistercard, and strip packaging. Strip packaging is a type of packaging wherein medications are packaged in individual pouches for administration on a specific date and, in some cases, at a specific time. Typically, individual pouches are removably joined together and often provided in rolls. The pouches can be separated from the roll when needed.

Drug product packaging systems typically include canisters that contain bulk quantities of a drug product to be packaged into a containment mechanism, such as a strip package, blistercard, pouch, or the like. Certain type of drug products, such as nutraceuticals, may be packaged in greater quantities than traditional prescription medications or drugs. Nutraceutical dispensers typically use a smaller formulary—less than 50 unique product—but dispense in the same high volume dispensing. As a result, it may be desirable to assign multiple canisters with the same drug product to increase the quantity of the drug product in the drug product packaging system that is available for packaging. More canisters of the same drug product type (NDC or SKEW) may improve the total output of the machine, reduce refill frequency, and stock outages.

### SUMMARY

In some embodiments of the inventive concept, a method comprises: generating an order for a drug product packaging system to dispense a quantity of drug product into respective ones of a plurality of pouches; associating a plurality of canisters in the drug product packaging system with each other as a group, each of the plurality of canisters containing the drug product; and operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

In other embodiments, the method further comprises determining that one of the plurality of canisters is empty when operating the drug product packaging system to dispense the quantity of drug product into one of the plurality of pouches; and refilling the plurality of canisters responsive to determining that the one of the plurality of canisters is empty.

In still other embodiments, the method further comprises operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.

In still other embodiments, the method further comprises maintaining the order without generating a new order for the drug product packaging system to dispense the quantity of drug product into the respective ones of the plurality of pouches in response to determining that the one of the plurality of canisters is empty.

In still other embodiments, the plurality of canisters comprises pinned canisters that are smart canisters.

In still other embodiments, the plurality of canisters comprises pinned canisters that are a mixture of smart and regular canisters.

In still other embodiments, a lot number of the drug product contained in a first one of the plurality of canisters is different from a lot number of the drug product contained in a second one of the plurality of canisters.

In still other embodiments, an expiration date of the drug product contained in a first one of the plurality of canisters is different from an expiration date of the drug product contained in a second one of the plurality of canisters.

In still other embodiments, a number of the plurality of canisters in the group is greater than eight and less than or equal to thirty-two.

In still other embodiments, the group is one of a plurality of canister grouping options in the drug product packaging system. The drug product packaging system is configured to perform different operations for dispensing drug product for different ones of the plurality of canister grouping options, respectively.

In still other embodiments, associating the plurality of canisters in the drug product packaging system with each other as the group comprises: receiving user input selecting the group from among the plurality of canister grouping options.

In still other embodiments, the drug product is a nutraceutical.

In some embodiments of the inventive concept, a system comprises: a processor; and a memory coupled to the processor and comprising computer readable program code embodied in the memory that is executable by the processor to perform operations comprising: generating an order for a drug product packaging system to dispense a quantity of drug product into respective ones of a plurality of pouches; associating a plurality of canisters in the drug product packaging system with each other as a group, each of the plurality of canisters containing the drug product; and operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

In further embodiments, the operations further comprise: determining that one of the plurality of canisters is empty when operating the drug product packaging system to dispense the quantity of drug product into one of the plurality of pouches; and refilling the plurality of canisters responsive to determining that the one of the plurality of canisters is empty.

In still further embodiments, the operations further comprise: operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.

In still further embodiments, the operations further comprise: maintaining the order without generating a new order for the drug product packaging system to dispense the quantity of drug product into the respective ones of the plurality of pouches in response to determining that the one of the plurality of canisters is empty.

In still further embodiments, the drug product is a nutraceutical.

In some embodiments of the inventive concept, a computer program product comprises: a non-transitory computer readable storage medium comprising computer readable program code embodied in the medium that is executable by a processor to perform operations comprising: generating an order for a drug product packaging system to dispense a quantity of drug product into respective ones of a plurality of pouches; associating a plurality of canisters in the drug product packaging system with each other as a group, each of the plurality of canisters containing the drug product; and operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

In other embodiments, the operations further comprise: determining that one of the plurality of canisters is empty when operating the drug product packaging system to dispense the quantity of drug product into one of the plurality of pouches; and refilling the plurality of canisters responsive to determining that the one of the plurality of canisters is empty.

In still other embodiments, the operations further comprise: operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a system for dispensing drug product in a drug product packaging system using round-robin drawn down from holding canisters in accordance with some embodiments of the inventive concept;

FIG. 2 is a flowchart that illustrates operations for dispensing drug product in the drug product packaging system of FIG. 1 in accordance with some embodiments of the inventive concept;

FIGS. 3A, 3B, and 3C are block diagrams that illustrate re-filling operations of the holding canisters in the drug product packaging system according to some embodiments of the inventive concept;

FIGS. 4 and 5 are flowcharts that illustrate further operations for dispensing drug product in the drug product packaging system of FIG. 1 in accordance with some embodiments of the inventive concept;

FIG. 6 is a block diagram that illustrates a user interface for selecting between holding canister grouping options in accordance with some embodiments of the inventive concept;

FIG. 7 is a data processing system that may be used to implement a packaging system interface server in the system for dispensing drug product in a drug product packaging system of FIG. 1 in accordance with some embodiments of the inventive concept; and

FIG. 8 is a block diagram that illustrates a software/hardware architecture for use in the system for dispensing drug product in a drug product packaging system of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components, and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “data processing facility” includes, but it is not limited to, a hardware element, firmware component, and/or software component. A data processing system may be configured with one or more data processing facilities.

The term “drug product packaging system,” as used herein, refers to any type of pharmaceutical dispensing system including, but not limited to, automated systems that fill vials, bottles, containers, pouches, blistercards, or the like with drug product, semi-automated systems that fill vials, bottles, containers, pouches, blistercards, strip packages, or the like with drug product, and any combination of automated and semi-automated systems for filling a drug product package with drug product. Drug product packaging system also includes packaging systems for pharmaceutical alternatives, such as nutraceuticals and/or bioceuticals.

The terms “pharmaceutical” and “medication,” as used herein, are interchangeable and refer to medicaments prescribed to patients either human or animal. A pharmaceutical or medication may be embodied in a variety of ways including, but not limited to, pill form capsule form, tablet form, and the like.

The term “drug product” refers to any type of medicament that can be packaged within a vial, bottle, container, pouch, blistercard, or the like by automated and semi-automated drug product packaging systems including, but not limited to, pills, capsules, tablets, caplets, gel caps, lozenges, and the like. Drug product also refers to pharmaceutical alternatives, such as nutraceuticals and/or bioceuticals. Example drug product packaging systems including management techniques for fulfilling packaging orders are described in U.S. Pat. No. 10,492,987 the disclosure of which is hereby incorporated herein by reference.

The term “drug product package” refers to any type of object that can hold a drug product including, but not limited to, a vial, bottle, container, pouch, blistercard, strip package, or the like.

The term “pouch” may be used to refer to a traditional pouch package containment mechanism as well as blistercard and strip package containment mechanisms.

Some embodiments of the inventive concept stem from a realization that certain types of drug products, such as nutraceuticals, may be packaged in greater quantities than traditional prescription medications or drugs. To facilitate packaging such drug products in greater quantities, multiple canisters that hold the drug product may be grouped together for dispensing therefrom. Two conventions for grouping holding canisters for dispensing drug product therefrom include what may be known as a cluster group and a firmware group. In a cluster group, multiple canisters may be associated with one another to form a group to dispense a particular drug product. These canisters may be smart canisters, which may be located anywhere in the drug product packaging system, regular canisters, which must be located in a specific spot. Canisters that are loaded, smart or regular, may be referred to as pinned canisters, which are assigned particular locations in the drug product packaging system. The canisters may respectively hold quantities of drug product corresponding to multiple lot numbers and expiration dates. The canisters are typically drawn from to fill pouches sequential order often based on expiration date of the drug product held therein. One drawback to the use of cluster groups, however, is that when a canister empties, the pouch that would have been filled based on the packaging order is left empty, a new order is generated, a new pouch is prepared for filling, and the new pouch is filled from the next canister in the sequence. This empty pouch requires personnel to remove the empty pouch from a roll of pouches and join the remaining pouches through a manual operation. The new pouch that is generated in place of the empty pouch may be called a “redo bag” and the empty pouch may be referred to as an “attention bag.” Correcting a roll of pouches in this manner may be labor intensive and increase the workload of support personnel significantly. Moreover, some implementations set limits on the maximum number of empty pouches or redo bags per roll of pouches.

The other convention for grouping canisters is known as a firmware group. This grouping convention avoids the problem of an empty pouch or redo bag by automatically proceeding to the next canister in the sequence when a canister empties. The firmware group, however, requires that the drug product held in the canisters in the group be from the same lot number and have the same expiration date. Moreover, due to firmware limitations in legacy drug product packaging systems, the number of canisters in a firmware group is limited to eight. This may result in more frequent refill operations and may be impractical for high volume drug product dispense orders, which may be more common with drug products, such as nutraceuticals.

Some embodiments of the inventive concept may provide a third convention for grouping of holding canisters for dispensing drug product therefrom and may be referred to as a level load group. In a level load group, a plurality of canisters may be associated with each other without the eight-canister limit associated with the firmware group convention. For example, thirty-two or more canisters may be associated with one another in a level load group, but embodiments of the inventive concept are not limited to a particular number. Rather than drawing from canisters sequentially, a drug product packaging system may draw from canisters in a level load group in round-robin fashion by switching canisters each time a new pouch is to be filled with that drug product. In contrast to the cluster group convention, when a canister is determined to be empty, all the canisters in the level load group are re-filled in parallel. This may significantly reduce the amount of time dedicating to re-filling empty canisters. In some drug product packaging systems, it can take up to twelve seconds or more of spinning or manipulating the canister without a drug product being dispensed to determine that a canister is empty. By re-filling all of the canisters in a level load group in parallel when a first canister in the group is determined to be empty, up to twelve seconds for each remaining canister in the level load group can be saved in the re-fill process. The drug product packaging system may also proceed with filling the current pouch once the refill process is completed without generating a new order. As a result, an empty pouch or redo bag may not be generated, which can eliminate the need for manual correction required for removal of the empty pouch associated with the cluster group convention. Similar to the cluster group convention, the level load group convention for grouping canisters may support smart canisters, and/or regular (not smart) canisters that are pinned, e.g., loaded into the drug product packaging system. The level load group convention does not require all drug product to be in the same lot and/or have the same expiration date. Thus, canisters in a level load group may contain drug product with the same or different lot numbers and/or expiration dates.

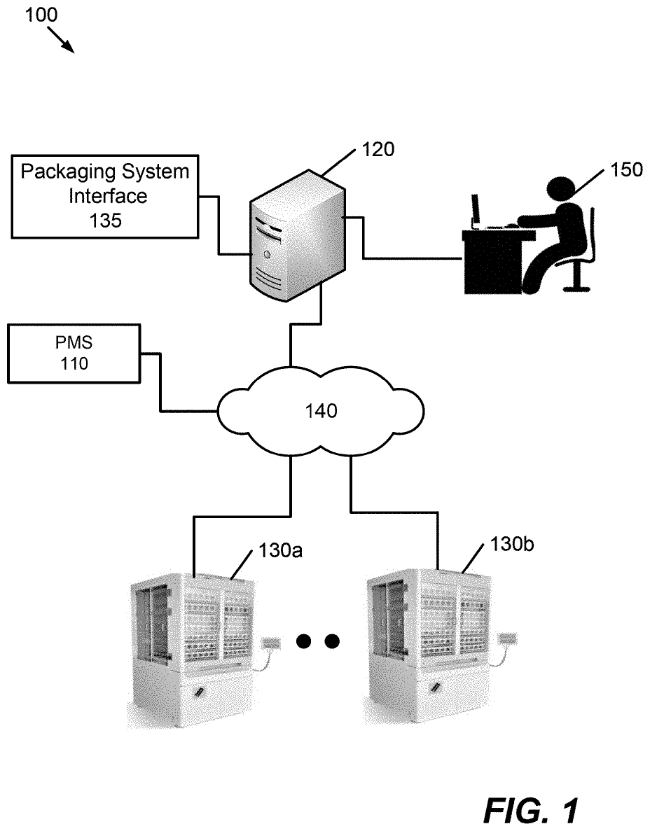

Referring to FIG. 1, a communication network 100 including a drug product packaging system that uses round-robin draw down from drug product holding canisters, in accordance with some embodiments of the inventive concept, comprises a pharmacy management system (PMS) or host system 110, a packaging system server 120, and one or more drug product packaging systems 130a and 130b that are coupled via a network 140 as shown.

The PMS system 110 may be configured to manage and fill prescriptions for customers. As used herein, PMS systems may be used in pharmacies or may be used generally as batch-generating systems for other applications, such as dispensing nutraceuticals or bioceuticals. The PMS system 110 may be associated with a variety of types of facilities, such as pharmacies, hospitals, long term care facilities, and the like. The PMS system or host system 110 may be any system capable of sending a valid prescription to the one or more product packaging systems 130a and 130b. The packaging system server 120 may include a packaging system interface module 135 and may be configured to manage the operation of the drug product packaging systems 130a and 130b. For example, the packaging system server 120 may be configured to receive packaging orders from the PMS system 110 and to identify which of the drug product packaging systems 130a and 130b should be used to package particular individual orders or batches of orders. In addition, the packaging system server 120 may be configured to manage the operations of the drug product packaging systems 130a and 130b. For example, the packaging system server 120 may be configured to manage the inventory of drug product available through each of the drug product packaging systems 130a and 130b, to manage the drug product dispensing canisters assigned or registered to one or more of the drug product packaging systems 130a and 130b, to manage the operational status generally of the drug product packaging systems 130a and 130b, and/or to manage reports regarding the status (e.g., assignment, completion, etc.) of packaging orders, drug product inventory, order billing, and the like. In some embodiments, the packaging system server may provide a user interface that may allow the canisters in one or more of the drug product packaging systems 130a and 130b to be grouped using a convention, such as the cluster group, level load group, or firmware group described above. A user 150, such as a pharmacist or pharmacy technician, may communicate with the packaging system server 120 using any suitable computing device via a wired and/or wireless connection and may control operations of the drug product packaging systems 130a and 130b through the user interface provided by the packaging system interface 135. Although the user 150 is shown communicating with the packaging system server 120 via a direct connection in FIG. 1, it will be understood that the user 150 may communicate with the packaging system server 120 via one or more network connections. The user 150 may interact with the packaging system server 120 to approve or override various recommendations made by the packaging system server 120 in operating the drug product packaging systems 130a and 130b. The user 150 may also initiate the running of various reports as described above for the drug product packaging systems 130a and 130b. Although only two drug product packaging systems 130a and 130b are shown in FIG. 1, it will be understood that more than two drug product packaging systems may be managed by the packaging system server 120.

In general, the drug product packaging systems 130a and 130b may be configured to package drug products using a variety of different containment types including, but not limited to, vials, bottles, containers, pouches, blistercards, strip packages, or the like. Embodiments will be described herein, however, with reference to the drug product packaging systems 130a and 130b being configured to package drug product using pouches as a containment mechanism, which includes blistercards and strip packages.

A network 140 couples the drug product packaging systems 130a and 130b, the PMS system 110, and the packaging system server 120 to one another. The network 140 may be a global network, such as the Internet or other publicly accessible network. Various elements of the network 140 may be interconnected by a wide area network, a local area network, an Intranet, and/or other private network, which may not be accessible by the general public. Thus, the communication network 140 may represent a combination of public and private networks or a virtual private network (VPN). The network 140 may be a wireless network, a wireline network, or may be a combination of both wireless and wireline networks.

Although FIG. 1 illustrates an example communication network that includes a drug product packaging system that uses round-robin draw down from drug product holding canisters, it will be understood that embodiments of the inventive subject matter are not limited to such configurations, but are intended to encompass any configuration capable of carrying out the operations described herein.

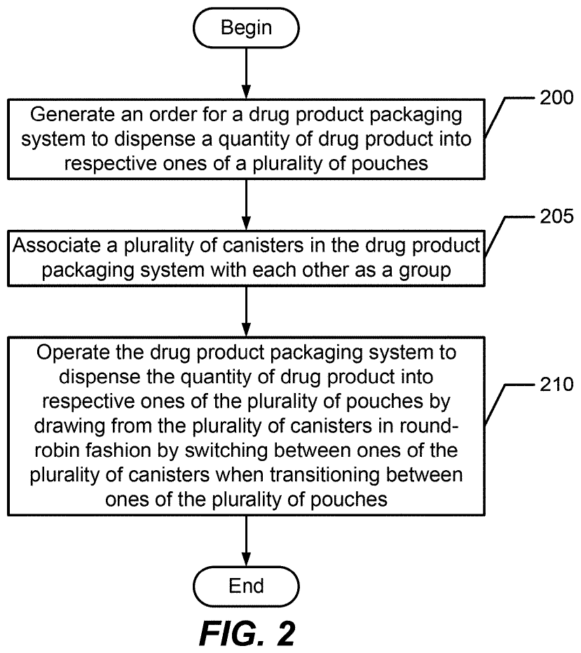

FIG. 2 is a flowchart that illustrates operations for dispensing drug product in the drug product packaging system. Referring now to FIG. 2, a drug product packaging order is generated using the PMS system 110 (block 200), which may be reviewed and processed by the packaging system server 120 before being communicated to one of the drug product packaging systems 130a or 130b for fulfillment. The order may include instructions to print patient and/or drug information on one or more pouches and to dispense a quantity of drug product into each of the one or more pouches. The canisters in the drug product packaging system 130a, 130b may be associated with each other as a group (block 205), such as a level load group as described above. The drug product packaging system 130a, 130b may operate to dispense the quantity of drug product into respective pouches by drawing from the canisters in the level-load group in round-robin fashion by switching between canisters when transitioning between pouches (block 210).

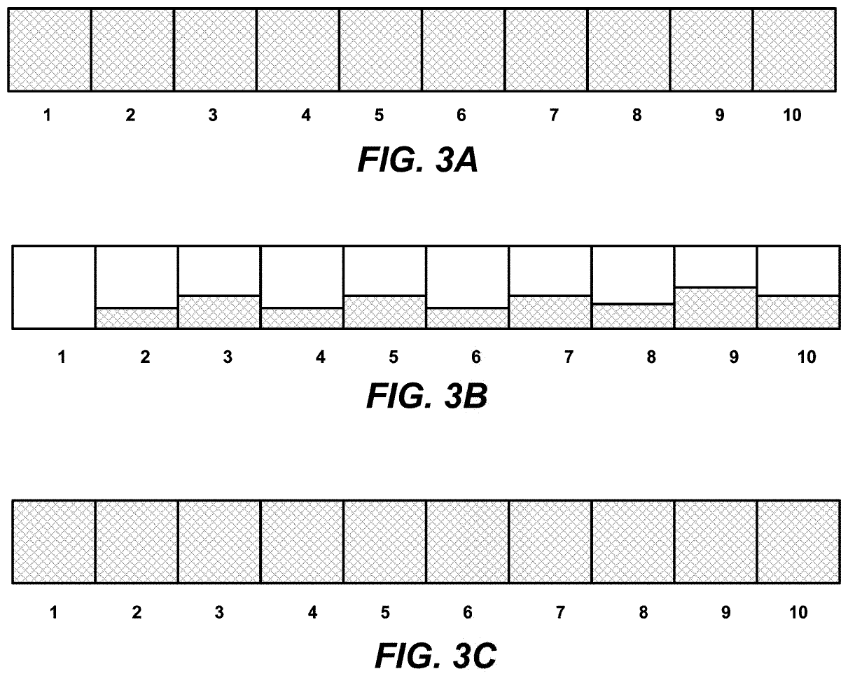

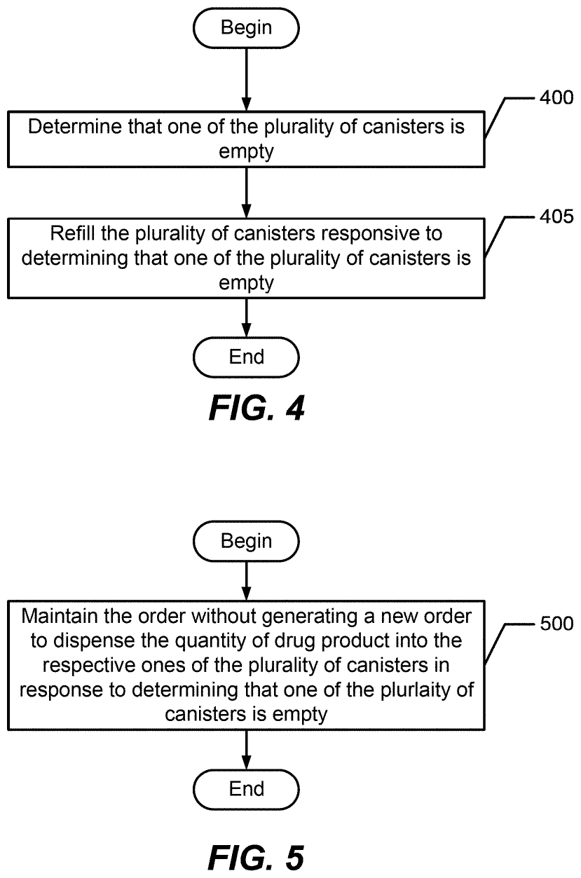

FIGS. 3A, 3B, and 3C are block diagrams that illustrate re-filling operations of the holding canisters in a level load group and FIGS. 4 and 5 are flowcharts that illustrate further operations for dispensing drug product in a drug product packaging system in accordance with some embodiments of the inventive concept. FIG. 3A is a block diagram that illustrates ten canisters in a level load group that are filled with drug product. As described above with respect to FIG. 2, drug product may be dispensed from these ten canisters in round-robin fashion by switching between the canisters when transitioning between pouches. Referring now to FIG. 4 and FIG. 3B, a determination is made that one of the canisters is empty (block 400). In the example of FIG. 3B, canister one empties first with the remaining canisters holding some drug product. Rather than just re-fill canister one, however, operations continue by re-filling all of the canisters in the level load group in parallel (block 405) as shown in FIG. 3C. Assuming a typical time of spinning or manipulating the canister for around twelve seconds to determine that the canister is empty, by re-filling all of the canisters in parallel, up to 108 seconds may be saved in the example of FIGS. 3A-3C in which ten canisters are assigned to a level load group by avoiding the emptying of each canister individually and then re-filling the individual canister.

Referring now to FIG. 5, in contrast with canisters in a cluster group, after determining that a canister is empty, dispensing of the drug product can resume after a re-fill operation of the canisters in a level load group is performed by maintaining the current drug product canister dispensing order without generating a new drug product dispensing order, which can result in the generation of an empty pouch or redo bag. This can eliminate the manual correction required to remove any empty pouches that are generated during the packaging process.

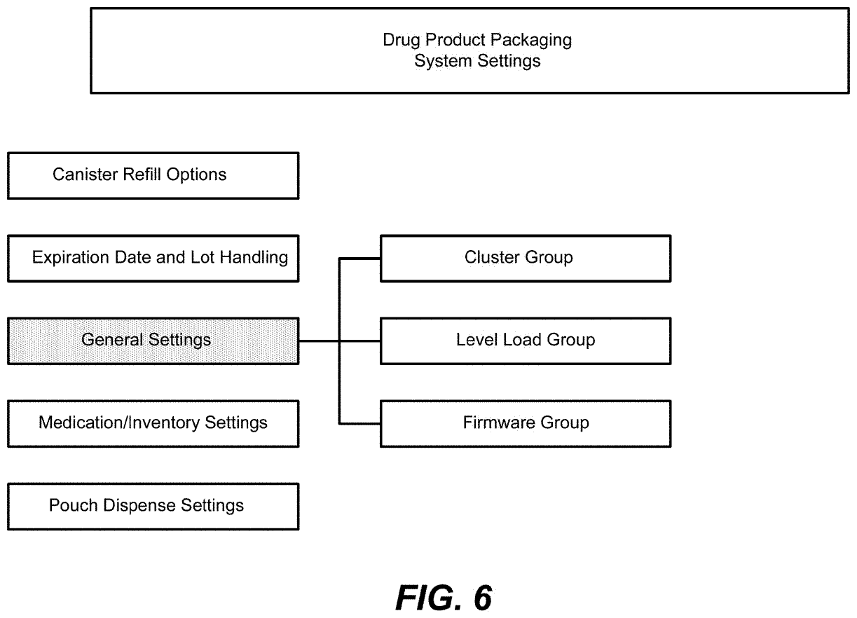

FIG. 6 is a block diagram that illustrates a user interface for selecting between holding canister grouping options in accordance with some embodiments of the inventive concept. The packaging system interface module 135 may provide a user interface that allows a user to control operation of the drug product packaging systems 130a, 130b including selecting between and defining various grouping conventions for canisters when dispensing drug product. As shown in FIG. 6, the user interface may include a drug product packaging system setting screen, which includes such items as “canister refill options,” “expiration date and lot handling,” “general settings,” “medication/inventory settings,” and “pouch dispense settings.” Under “general settings,” a user may select one of the canister grouping conventions including the “cluster group” convention, “level load group” convention, and the “firmware group” convention. A user may, therefore, select the “level load group” convention to associate a plurality of canisters in a level load group to dispense drug product therefrom in round-robin fashion as described above and to re-fill the canisters in the group in parallel the first time one of the canisters in the group is determined to be empty thereby saving re-fill time compared to individual or serial re-filling of canisters associated with the cluster group and firmware group conventions. Furthermore, an empty pouch or redo bag is not generated upon detecting an empty canister, which is characteristic of the cluster group convention.

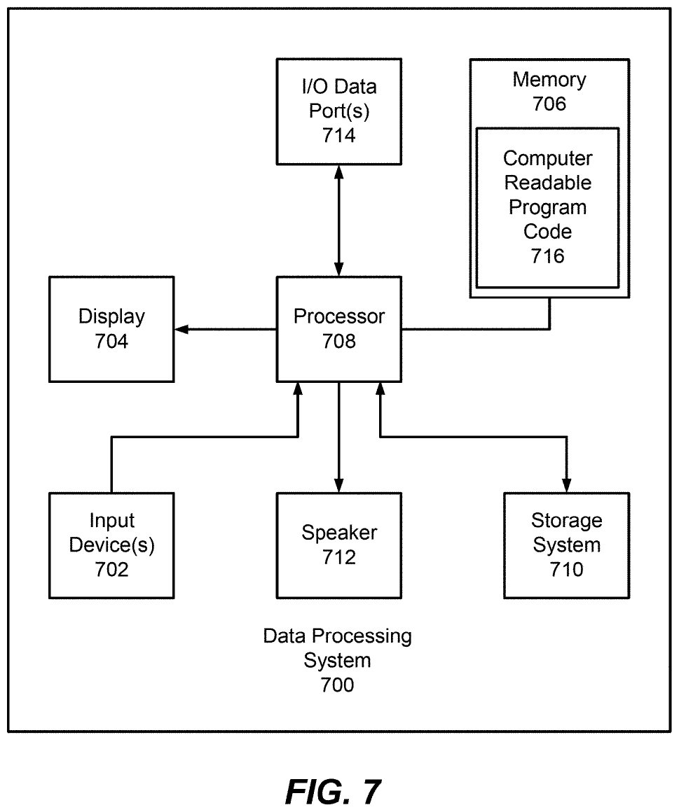

Referring now to FIG. 7, a data processing system 700 that may be used to implement the packaging system server 120 of FIG. 1, in accordance with some embodiments of the inventive concept, comprises input device(s) 702, such as a keyboard or keypad, a barcode scanner, or RFID reader, a display 704, and a memory 706 that communicates with a processor 708. The data processing system 700 may further include a storage system 710, a speaker 712, and an input/output (I/O) data port(s) 714 that also communicate with the processor 708. The processor 708 may be, for example, a commercially available or custom microprocessor. The storage system 710 may include removable and/or fixed media, such as floppy disks, ZIP drives, hard disks, or the like, as well as virtual storage, such as a RAMDISK. The I/O data port(s) 714 may be used to transfer information between the data processing system 1100 and another computer system or a network (e.g., the Internet). These components may be conventional components, such as those used in many conventional computing devices, and their functionality, with respect to conventional operations, is generally known to those skilled in the art. The memory 706 may be configured with computer readable program code 716 to facilitate dispensing drug product in a drug product packaging system using round-robin draw down from canisters using a level loading group convention according to some embodiments of the inventive concept.

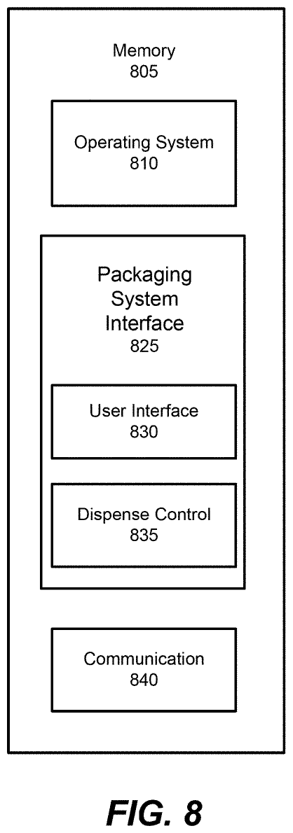

FIG. 8 illustrates a memory 805 that may be used in embodiments of data processing systems, such as the packaging system server 120 of FIG. 1 and the data processing system 700 of FIG. 7, respectively, to facilitate dispensing drug product in a drug product packaging system using round-robin draw down from canisters using a level loading group convention according to some embodiments of the inventive concept. The memory 805 is representative of the one or more memory devices containing the software and data used for facilitating operations of the packaging system server 120 and the packaging system interface 135 as described herein. The memory 805 may include, but is not limited to, the following types of devices: cache, ROM, PROM, EPROM, EEPROM, flash, SRAM, and DRAM. As shown in FIG. 8, the memory 805 may contain three or more categories of software and/or data: an operating system 810, a packaging system interface module 825, and a communication module 840. In particular, the operating system 810 may manage the data processing system's software and/or hardware resources and may coordinate execution of programs by the processor. The packaging system interface module 825 may include a user interface module 830 and a dispense control module 835. The user interface module may be configured to perform one or more operations described above with respect to FIG. 6. The dispense control module 835 may be configured to perform one or more operations described above with respect to the flowcharts of FIGS. 2, 4, and 5 and the block diagrams of FIGS. 3A-3C. The communication module 840 may be configured to support communication between, for example, packaging system server 120 and the PMS 110 and between the packaging system server 120 and the drug product packaging systems 130a, 130b.

Although FIG. 8 illustrates a hardware/software architecture that may be used in data processing systems, such as the packaging system server 120 of FIG. 1 and the data processing system 700 of FIG. 7, respectively, in accordance with some embodiments of the inventive concept, it will be understood that embodiments of the present invention are not limited to such a configuration but are intended to encompass any configuration capable of carrying out operations described herein.

Computer program code for carrying out operations of data processing systems discussed above with respect to FIGS. 1-7 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the packaging system server 120 of FIG. 1 and the data processing system 700 of FIG. 7 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-8 may be used to facilitate dispensing of drug product in a drug product packaging system using round-robin drawn down from holding canisters in a level load group according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 805 when coupled to a processor includes computer readable program code that, when executed by the processor, causes the processor to perform operations including one or more of the operations described herein with respect to FIGS. 1-6.

As described above, embodiments of the inventive concept may provide a convention for grouping holding canisters for dispensing drug product therefrom, which is referred to as a level load group. Drug product may be dispensed from canisters in a level load group in round-robin fashion and re-filled in parallel the first time one of the canisters in the group is determined to be empty. This may save time in refilling canisters serially as they empty. The canisters may also contain drug product from different lot numbers and with different expiration dates providing more flexibility than conventions for grouping canisters, such as a firmware group, which require all drug product in the canister grouping to have the same lot number and expiration date. Moreover, the level load group convention does not limit the number of canisters in the group to eight, a limitation of the firmware group convention, and does not require the generation of a new order upon detecting an empty canister, a characteristic of the cluster group convention, which results in the generation of an empty pouch or redo bag requiring manual correction for removal of the empty pouch.

### Further Definitions and Embodiments

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises,” “comprising,” “include”, “including”, “includes”, “have”, “has”, “having”, or variants thereof when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

It will also be understood that, although the terms first, second, etc. may be used herein to describe various elements, these elements should not be limited by these terms. These terms are only used to distinguish one element from another.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

What is claimed is:

1. A method comprising:
generating an order for a drug product packaging system to dispense a quantity of drug product into respective ones of a plurality of pouches;
associating a plurality of canisters in the drug product packaging system with each other as a group, each of the plurality of canisters containing the drug product; and
operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

2. The method of claim 1, further comprising:
determining that one of the plurality of canisters is empty when operating the drug product packaging system to dispense the quantity of drug product into one of the plurality of pouches; and
refilling the plurality of canisters responsive to determining that the one of the plurality of canisters is empty.

3. The method of claim 2, further comprising:
operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.

4. The method of claim 2, further comprising:
maintaining the order without generating a new order for the drug product packaging system to dispense the quantity of drug product into the respective ones of the plurality of pouches in response to determining that the one of the plurality of canisters is empty.

5. The method of claim 1, wherein the plurality of canisters comprises pinned cannisters that are smart canisters.

6. The method of claim 1, wherein the plurality of canisters comprises pinned canisters that are a mixture of smart canisters and regular canisters.

7. The method of claim 1, wherein a lot number of the drug product contained in a first one of the plurality of canisters is different from a lot number of the drug product contained in a second one of the plurality of canisters.

8. The method of claim 1, wherein an expiration date of the drug product contained in a first one of the plurality of canisters is different from an expiration date of the drug product contained in a second one of the plurality of canisters.

9. The method of claim 1, wherein a number of the plurality of canisters in the group is greater than eight and less than or equal to thirty-two.

10. The method of claim 1, wherein the group is one of a plurality of canister grouping options in the drug product packaging system; and
wherein the drug product packaging system is configured to perform different operations for dispensing drug product for different ones of the plurality of canister grouping options, respectively.

11. The method of claim 10, wherein associating the plurality of canisters in the drug product packaging system with each other as the group comprises:
receiving user input selecting the group from among the plurality of canister grouping options.

12. The method of claim 1, wherein the drug product is a nutraceutical.

13. A system, comprising:
a processor; and
a memory coupled to the processor and comprising computer readable program code embodied in the memory that is executable by the processor to perform operations comprising:
generating an order for a drug product packaging system to dispense a quantity of drug product into respective ones of a plurality of pouches;
associating a plurality of canisters in the drug product packaging system with each other as a group, each of the plurality of canisters containing the drug product; and
operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

14. The system of claim 13, wherein the operations further comprise:
determining that one of the plurality of canisters is empty when operating the drug product packaging system to dispense the quantity of drug product into one of the plurality of canisters; and
refilling the plurality of canisters responsive to determining that the one of the plurality of canisters is empty.

15. The system of claim 14, wherein the operations further comprise:
operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.

16. The system of claim 14, wherein the operations further comprise:
maintaining the order without generating a new order for the drug product packaging system to dispense the quantity of drug product into the respective ones of the plurality of pouches in response to determining that the one of the plurality of canisters is empty.

17. The system of claim 13, wherein the drug product is a nutraceutical.

18. A computer program product, comprising:
a non-transitory computer readable storage medium comprising computer readable program code embodied in the medium that is executable by a processor to perform operations comprising:
generating an order for a drug product packaging system to dispense a quantity of drug product into respective ones of a plurality of pouches;
associating a plurality of canisters in the drug product packaging system with each other as a group, each of the plurality of canisters containing the drug product; and
operating the drug product packaging system to dispense the quantity of drug product into respective ones of the plurality of pouches by drawing from the plurality of canisters in round-robin fashion by switching between ones of the plurality of canisters when transitioning between ones of the plurality of pouches.

19. The computer program product of claim 18, wherein the operations further comprise:
determining that one of the plurality of canisters is empty when operating the drug product packaging system to dispense the quantity of drug product into one of the plurality of pouches; and
refilling the plurality of canisters responsive to determining that the one of the plurality of canisters is empty.

20. The computer program product of claim 19, wherein the operations further comprise:
operating the drug product packaging system to dispense the quantity of drug product into the respective ones of the plurality of pouches by drawing from the plurality of canisters in round-robin fashion by switching between the ones of the plurality of canisters when transitioning between the ones of the plurality of pouches responsive to refilling the plurality of canisters based on the order.
